(19) United States
(12) Patent Application Publication
(10) Pub. No.: US 2022/0096475 A1
(43) Pub. Date: Mar. 31, 2022

(54) METHOD FOR TREATING SARCOIDOSIS-ASSOCIATED PULMONARY HYPERTENSION

(71) Applicant: ALTAVANT SCIENCES GmbH, Basel (CH)

(72) Inventors: Thomas PACK, Durham, NC (US); Sudarshan Rajagopal, Chapel Hill, NC (US)

(73) Assignee: ALTAVANT SCIENCES GmbH, Basel (CH)

(21) Appl. No.: 17/507,295

(22) Filed: Oct. 21, 2021

Related U.S. Application Data
(63) Continuation of application No. 16/714,227, filed on Dec. 13, 2019, now abandoned.
(60) Provisional application No. 62/780,732, filed on Dec. 17, 2018.

(51) Int. Cl. A61K 31/506; A61P 9/12

(52) U.S. Cl. CPC A61K 31/506; A61P 9/12

(57) ABSTRACT

There is a method of treating or preventing sarcoidosis-associated pulmonary hypertension in a patient. The method has the step of administering to the patient a therapeutically effective amount of one or more compounds: (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate or a pharmaceutically acceptable salt thereof, or (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid or a pharmaceutically acceptable salt thereof, or a combination of the foregoing.

### CROSS-REFERENCE TO A RELATED APPLICATION

The present application is a continuation application of U.S. Ser. No. 16/714,227, filed Dec. 13, 2019, which claims priority to U.S. Provisional Application No. 62/780,732, filed Dec. 17, 2018, the contents of both of which are incorporated herein in its entirety via incorporation by reference.

### BACKGROUND OF THE DISCLOSURE

### 1. Field of the Disclosure

The present disclosure relates to a method for treating sarcoidosis-associated pulmonary hypertension (SAPH) in a patient.

### 2. Description of the Prior Art

Pulmonary hypertension (PAH) is one form of a broader condition known as pulmonary hypertension, which means high blood pressure in the lungs. In PAH, increased pressure in the vessels is caused by obstruction in small arteries in the lungs and/or constriction or narrowing in diameter, which increases the resistance to blood flow through the lungs. Over time, the increased blood pressure can damage the heart. In many cases of pulmonary hypertension, the cause is unknown. Others causes can be drug-related, HIV infection, and connective tissue/autoimmune disorders (such as scleroderma).

There are currently five accepted classifications of pulmonary hypertension by the World Symposium on Pulmonary Hypertension as described in the scientific literature: BMJ, 36:j5492 (2018), “Pulmonary hypertension: pathogenesis and clinical management”. Class 5 includes Pulmonary Hypertension with unclear and/or multifactorial mechanisms. Within class 5, a subset of pulmonary hypertension is described that consists of pulmonary hypertension due to systemic disorders of which sarcoidosis is a listed disorder known to cause pulmonary hypertension. Sarcoidosis has an unknown etiology and leads to formation of granulomas due to improper response of immune cells to antigens. Granulomas appear in the lungs in most sarcoidosis patients. Granulomatous inflammation of the pulmonary vessels results in compression of pulmonary arteries which can lead to the formation of pulmonary hypertension. Granulomas eventually result in fibrosis and loss of function in the lungs.

Serotonin (5-hydroxytryptamine, 5-HT) is a hormone that modulates central and peripheral functions by acting on neurons, smooth muscle, and other cell types. 5-HT is involved in the control and modulation of multiple physiological and psychological processes, including in lung and pulmonary diseases. The literature discloses the relationship between 5-HT and pulmonary diseases at PloS One 7, e31617 (2012), “The Role of Circulating Serotonin in the Development of Chronic Obstructive Pulmonary Disease” and Thorax 1999, 54, 161-168, “Role of Serotonin in the Pathogenesis of Acute and Chronic Pulmonary Hypertension”.

The rate-limiting step in 5-HT biosynthesis is the hydroxylation of tryptophan by dioxygen, which is catalyzed by tryptophan hydroxylase (TPH; EC 1.14.16.4) in the presence of the cofactor (6R)-L-erythro-5,6,7,8-tetrahydrobiopterin (BH4). The resulting oxidized product, 5-hydroxy tryptophan (5-HTT) is subsequently decarboxylated by an aromatic amino acid decarboxylase (AAAD; EC 4.1.1.28) to produce 5-HT. Together with phenylalanine hydroxylase (PheOH) and tyrosine hydroxylase (TH), TPH belongs to the pterin-dependent aromatic amino acid hydroxylase family.

Two vertebrate isoforms of TPH, namely TPH1 and TPH2, have been identified. TPH1 is primarily expressed in the pineal gland and non-neuronal tissues, such as enterochromaffin (EC) cells located in the gastrointestinal (GI) tract. TPH2 (the dominant form in the brain) is expressed exclusively in neuronal cells, such as dorsal raphe or myenteric plexus cells. The peripheral and central systems involved in 5-HT biosynthesis are isolated, with 5-HT being unable to cross the blood-brain barrier. Therefore, the pharmacological effects of 5-HT can be modulated by agents affecting TPH in the periphery, mainly TPH1 in the gut or through agents that cannot efficiently cross the blood-brain barrier to affect central production of 5-HT.

WO 2015/035113 and U.S. Pat. No. 9,199,994 disclose spirocyclic compounds that act as inhibitors of THP and are useful in the treatment of certain various diseases and disorders associated with peripheral serotonin, namely, cardiovascular diseases of pulmonary hypertension (PAH) and associated pulmonary hypertension (APAH).

There is a need for a method of treating or preventing SAPH in a patient.

### SUMMARY OF THE DISCLOSURE

According to the present disclosure, there is provided a method of treating or preventing sarcoidosis-associated pulmonary hypertension (SAPH) in a patient. The method has the step of administering to the patient a therapeutically effective amount of a compound selected from the group consisting of (i) (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate, (ii) (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid, and (iii) a combination of the foregoing.

According to the present disclosure, there is provided a method of treating or preventing SAPH in a patient. The method has the step of administering to the patient a therapeutically effective amount of a composition including a compound selected from the group consisting of (i) (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate, (ii) (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid, and (iii) a combination of the foregoing, together with one or more pharmaceutically acceptable excipients.

According to the present disclosure, there is provided a method of treating or preventing SAPH in a patient. The method has the step of administering to the patient a composition including a therapeutically effective amount of a compound selected from the group consisting of (i) (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate or a pharmaceutically acceptable salt thereof, (ii) (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid or a pharmaceutically acceptable salt thereof, and (iii) a combination of the foregoing and a pharmaceutically acceptable excipient.

According to the present disclosure, there is provided the use of a therapeutically effective amount of a compound in treating SAPH. The compound is selected from the group consisting of (i) (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate or a pharmaceutically acceptable salt thereof, (ii) (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid or a pharmaceutically acceptable salt thereof, and (iii) a combination of the foregoing.

### BRIEF DESCRIPTION OF THE DRAWINGS

Embodiments of the present disclosure are described herein with reference to the following figures.

FIG. 1 is a plot of an XRPD of a crystalline compound of (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate according to the present disclosure (crystalline Form 3).

FIG. 2 is a plot of an XRPD of a crystalline compound of (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate of a different polymorphic form than that of FIG. 1 (crystalline Form 1).

### DETAILED DESCRIPTION OF THE DISCLOSURE

Sarcoidosis-associated pulmonary hypertension (SAPH) is treated via administration of either or both of two spirocyclic compounds. Without being bound to any theory, each of the two spirocyclic compounds act to inhibit the formation of 5-HT, and, thus, diminish or ameliorate effects of SAPH.

A useful spirocyclic compound is (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate (also referred to herein as “Compound A”). The compound has the following formula:

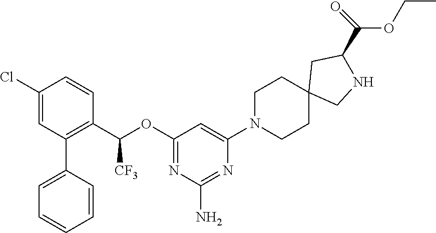 [SMILES: CCOC(=O)[C@@H]1CC2(CCN(c3cc(O[C@H](c4ccc(Cl)cc4-c4ccccc4)C(F)(F)F)nc(N)n3)CC2)CN1]

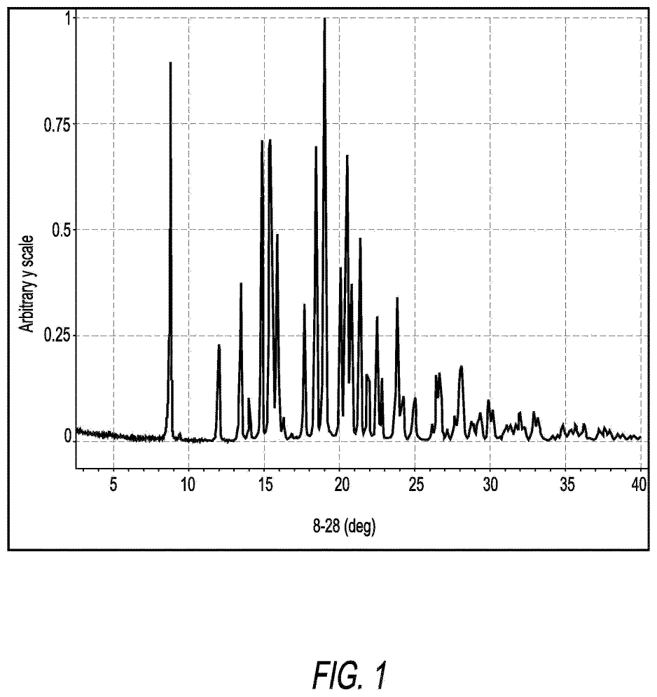

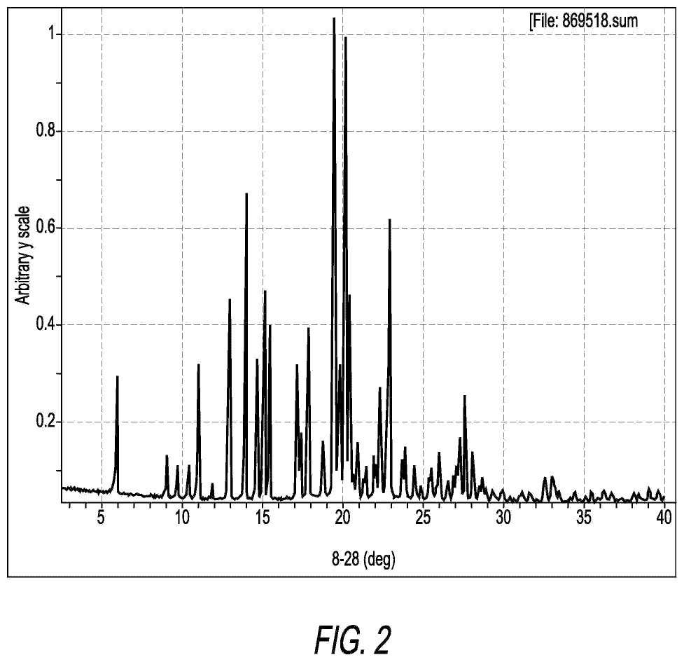

The above-referenced compound can be used to prevent or treat SAPH in either an amorphous or crystalline form. Two different polymorph crystalline forms are identified by x-ray powder diffraction patterns set forth in FIG. 1 (Form 3) and FIG. 2 (Form 1) as well as Tables 1 and 2 (Form 3) and Tables 3 and 4 (Form 1). The crystalline Form 3 polymorph might be preferred, as it exhibits substantially greater stability and shelf life compared to the crystalline Form 1 polymorph, particularly at temperatures of less than 95° C.

TABLE 1
Observed Peaks for X-ray Powder Diffraction
Pattern for Compound A, Crystalline Form 3

| Peak position (°2θ) | d space (Å) | Intensity (%) |
|---|---|---|
| 8.78 ± 0.20 | 10.077 ± 0.235 | 90 |
| 12.00 ± 0.20 | 7.375 ± 0.125 | 25 |
| 13.47 ± 0.20 | 6.573 ± 0.099 | 39 |
| 14.02 ± 0.20 | 6.316 ± 0.091 | 12 |
| 14.87 ± 0.20 | 5.956 ± 0.081 | 71 |
| 15.39 ± 0.20 | 5.757 ± 0.075 | 72 |
| 15.61 ± 0.20 | 5.677 ± 0.073 | 78 |
| 15.89 ± 0.20 | 5.576 ± 0.071 | 50 |
| 16.31 ± 0.20 | 5.434 ± 0.067 | 7 |
| 17.70 ± 0.20 | 5.011 ± 0.057 | 34 |
| 18.45 ± 0.20 | 4.809 ± 0.052 | 70 |
| 19.05 ± 0.20 | 4.658 ± 0.049 | 100 |
| 20.12 ± 0.20 | 4.413 ± 0.044 | 42 |
| 20.57 ± 0.20 | 4.317 ± 0.042 | 68 |
| 20.84 ± 0.20 | 4.262 ± 0.041 | 39 |
| 21.46 ± 0.20 | 4.141 ± 0.039 | 49 |
| 21.94 ± 0.20 | 4.051 ± 0.037 | 18 |
| 22.56 ± 0.20 | 3.941 ± 0.035 | 31 |
| 22.90 ± 0.20 | 3.884 ± 0.034 | 17 |
| 23.90 ± 0.20 | 3.723 ± 0.031 | 35 |
| 24.32 ± 0.20 | 3.660 ± 0.030 | 13 |
| 25.07 ± 0.20 | 3.552 ± 0.028 | 12 |
| 26.54 ± 0.20 | 3.359 ± 0.025 | 17 |
| 26.76 ± 0.20 | 3.332 ± 0.025 | 18 |
| 27.79 ± 0.20 | 3.210 ± 0.023 | 8 |
| 28.21 ± 0.20 | 3.163 ± 0.022 | 19 |
| 29.48 ± 0.20 | 3.030 ± 0.020 | 9 |

TABLE 2
Prominent Observed Peaks for X-ray Powder Diffraction
Pattern for Compound A, Crystalline Form 3

| Peak position (°2θ) | d space (Å) | Intensity (%) |
|---|---|---|
| 8.78 ± 0.20 | 10.077 ± 0.235 | 90 |
| 14.87 ± 0.20 | 5.956 ± 0.081 | 71 |
| 15.39 ± 0.20 | 5.757 ± 0.075 | 72 |
| 15.61 ± 0.20 | 5.677 ± 0.073 | 78 |
| 18.45 ± 0.20 | 4.809 ± 0.052 | 70 |
| 19.05 ± 0.20 | 4.658 ± 0.049 | 100 |

In yet another aspect, the Form 3 crystalline polymorph exhibits a characteristic XRPD peak at 19.05±0.20 (°2θ).

The Form 1 crystalline compound exhibits the XRPD (X-ray powder diffraction) pattern set forth below in Table 3.

TABLE 3
Observed Peaks for X-Ray Powder Diffraction
Pattern for Compound A, Crystalline Form 1

| Peak position (°2θ) | d space (Å) | Intensity (%) |
|---|---|---|
| 5.92 ± 0.20 | 14.936 ± 0.522 | 27 |
| 9.01 ± 0.20 | 9.816 ± 0.222 | 11 |
| 9.68 ± 0.20 | 9.140 ± 0.192 | 9 |
| 10.38 ± 0.20 | 8.523 ± 0.167 | 9 |
| 10.95 ± 0.20 | 8.082 ± 0.150 | 30 |
| 11.85 ± 0.20 | 7.468 ± 0.128 | 6 |
| 12.90 ± 0.20 | 6.861 ± 0.108 | 43 |
| 13.89 ± 0.20 | 6.376 ± 0.093 | 65 |
| 14.62 ± 0.20 | 6.057 ± 0.084 | 31 |
| 15.04 ± 0.20 | 5.890 ± 0.079 | 44 |
| 15.41 ± 0.20 | 5.750 ± 0.075 | 38 |
| 17.13 ± 0.20 | 5.176 ± 0.061 | 30 |
| 17.83 ± 0.20 | 4.974 ± 0.056 | 37 |
| 18.72 ± 0.20 | 4.741 ± 0.051 | 14 |
| 19.44 ± 0.20 | 4.567 ± 0.047 | 100 |
| 19.79 ± 0.20 | 4.487 ± 0.045 | 30 |
| 20.11 ± 0.20 | 4.417 ± 0.044 | 97 |
| 20.34 ± 0.20 | 4.366 ± 0.043 | 44 |
| 20.84 ± 0.20 | 4.262 ± 0.041 | 14 |
| 21.41 ± 0.20 | 4.151 ± 0.039 | 10 |
| 21.88 ± 0.20 | 4.063 ± 0.037 | 11 |
| 22.28 ± 0.20 | 3.991 ± 0.036 | 25 |
| 22.83 ± 0.20 | 3.895 ± 0.034 | 60 |
| 23.85 ± 0.20 | 3.731 ± 0.031 | 13 |
| 24.40 ± 0.20 | 3.648 ± 0.030 | 9 |
| 25.45 ± 0.20 | 3.500 ± 0.027 | 9 |
| 25.97 ± 0.20 | 3.431 ± 0.026 | 12 |
| 27.22 ± 0.20 | 3.276 ± 0.024 | 15 |
| 27.58 ± 0.20 | 3.235 ± 0.023 | 23 |
| 28.06 ± 0.20 | 3.180 ± 0.022 | 12 |
| 28.66 ± 0.20 | 3.115 ± 0.021 | 7 |

In still another aspect, the Form 1 crystalline compound exhibits prominent XRPD peaks set forth below in Table 4.

TABLE 4
Prominent Observed Peaks for X-Ray Powder Diffraction
Pattern for Compound A, Crystalline Form 1

| Peak position (°2θ) | d space (Å) | Intensity (%) |
|---|---|---|
| 12.90 ± 0.20 | 6.861 ± 0.108 | 43 |
| 13.89 ± 0.20 | 6.376 ± 0.093 | 65 |
| 15.04 ± 0.20 | 5.890 ± 0.079 | 44 |
| 19.44 ± 0.20 | 4.567 ± 0.047 | 100 |
| 20.11 ± 0.20 | 4.417 ± 0.044 | 97 |
| 20.34 ± 0.20 | 4.366 ± 0.043 | 44 |
| 22.83 ± 0.20 | 3.895 ± 0.034 | 60 |

The amorphous form of the Compound A can be prepared by the method set forth in Example 63i of U.S. Pat. No. 9,199,994 (corresponding to WO2015/054202), which is incorporated by reference herein in its entirety. The amorphous form can then be converted to crystalline form by extraction with organic solvents, such as C4 to C10 alcohols, C4 to C10 alkyl acetates, and ethers. Useful alcohols include pentane, hexane, and heptane. A useful ether is methyl tert butyl ether (MTBE). By way of example, crystalline form 1 can be prepared by extraction with isopropanol, ethanol, cyclohexane, ethyl acetate, acetone, water, and mixtures of the foregoing, while crystalline Form 3 can be prepared by extraction with MTBE and/or heptane. Crystalline Forms 1 and 3 can be prepared by the extraction techniques set forth in U.S. Provisional Application No. 62/767,171, filed Nov. 14, 2018, which is incorporated by reference herein in its entirety.

Another useful spirocyclic compound is (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid (herein alternately referred to as “Compound B”) of the following formula:

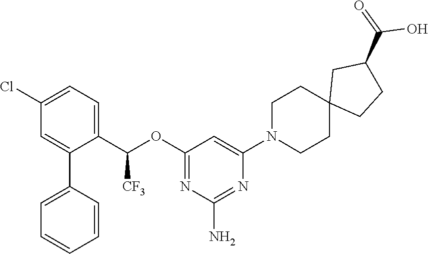 [SMILES: Nc1nc(O[C@H](c2ccc(Cl)cc2-c2ccccc2)C(F)(F)F)cc(N2CCC3(CC[C@H](C(=O)O)C3)CC2)n1]

The amorphous form of the Compound B can be prepared by the method set forth in Example 34c of U.S. Pat. No. 9,199,994.

The efficacy of amorphous (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate in inhibiting TPH1 in mice was demonstrated in U.S. Pat. No. 9,199,994 in biological assays at Example 63i and Table 27.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” is used interchangeably, refers to any animal, including mammals, preferably mice, rats, other rodents, rabbits, dogs, cats, swine, cattle, sheep, horses, or primates, and, most preferably, humans. Humans are inclusive of humans of any age, including adults and children, including infants.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” refers to reducing risk incidence, delaying, or inhibiting the onset or worsening of the disease; for example, in an individual who may be predisposed to the disease, condition or disorder but does not yet experience or display the pathology or symptomatology of the disease, or an individual who has previously suffered from the disease, condition or disorder, but has been treated and, e.g., no longer displays the pathology or symptomatology of the disease.

The spirocyclic compounds can be administered to patients (animals or humans) in need of such treatment in appropriate dosages that will provide prophylactic and/or therapeutic efficacy. The dose required for use in the treatment or prevention of any particular disease or disorder will typically vary from patient to patient depending on, for example, particular compound or composition selected, the route of administration, the nature of the condition being treated, the age and condition of the patient, concurrent medication or special diets then being followed by the patient, and other factors. The appropriate dosage can be determined by the treating physician; however, the dosage will be within the parameters defined herein.

The spirocyclic compounds can be administered systemically orally, subcutaneously, parenterally, by inhalation/sufflation or rectally in dosage unit compositions containing pharmaceutically acceptable carriers, adjuvants and vehicles. Parenteral administration can involve subcutaneous injections, intravenous or intramuscular injections or infusion techniques. Injectable liquids can include aqueous and/or organic components. Treatment duration can be as long as deemed necessary by a treating physician. The compositions can be administered as often as needed, e.g., one to four (or more) or more times per day. Administration can take place twice daily, thrice daily, daily, weekly, biweekly, twice weekly, every other week, monthly, and the like. A treatment period can terminate when a desired result, for example, a particular therapeutic effect, is achieved. However, in some instances, a treatment period can be continued indefinitely.

In some embodiments, pharmaceutical compositions can be prepared as solid dosage forms for oral administration (e.g., capsules, tablets, pills, dragees, powders, granules and the like). Tablets can be prepared by compression and/or molding. Compressed tablets can include pharmaceutically acceptable excipients, such as, but not limited to, binders, lubricants, glidants, inert diluents, preservatives, disintegrants, and dispersing agents. Tablets and other solid dosage forms, such as, but not limited to, capsules, pills, powders, and granules, can include coatings, such as enteric coatings.

Liquid dosage forms for oral administration can include, for example, vehicles such as, but not limited to, pharmaceutically acceptable emulsions, microemulsions, solutions, suspensions, syrups and elixirs. The liquid vehicles can have aqueous and/or organic components. Suspensions can include one or more suspending agents. Examples of ingredients useful in liquid dosage forms include, but are not limited to, chelants, sequestering agents, viscosifiers, thickeners, penetration enhancers, solvents, emulsifiers, and emollients.

Pharmaceutical compositions suitable for parenteral administration can include the spirocyclic compound together with one or more pharmaceutically acceptable sterile isotonic aqueous or non-aqueous solutions, dispersions, suspensions or emulsions.

Alternatively, the composition can be in the form of a sterile powder that can be reconstituted into a sterile injectable solutions or dispersion just prior to use.

Solid and liquid dosage forms can be formulated such that they conform to a desired release profile, e.g., immediate release, delayed release, and extended or sustained release.

The amount of spirocyclic compound to be administered will vary depending on factors such as the following: the spirocyclic compound selected, method of administration, release profile, and composition formulation. Typically, for the Form 3 spirocyclic compound in an oral dosage form to treat or prevent a disease, particularly PH/PAH/APAH/IPAH/FPAH, a typical dosage will be about 1 mg/kg/day to about 50 mg/kg/day and more typically from about 5 mg/kg/day to about 30 mg/kg/day, based on the weight of the patient. A most preferred spirocyclic compound is RVT-1201 in crystalline Form 3. Individual oral dosage forms typically have from about 50 mg to about 3000 mg of a spirocyclic compound and additional amounts of one or more pharmaceutically acceptable excipients. Other useful individual oral dosage forms can, by way of example, have spirocyclic compound in amounts of 100 mg, 150 mg, 200 mg, 250 mg, 300 mg, 350 mg, or 400 mg, 450 mg, 500 mg, 550 mg, 575 mg, 600 mg, 625 mg, 650 mg, 675 mg, 700 mg, 725 mg, 750 mg, 775 mg, 800 mg, 900 mg, 950 mg, 1000 mg, 1050 mg, 1100 mg, 1150 mg, and about 1200 mg, particularly 1200 mg. A preferred dosage is 1200 mg. Other amounts between 50 mg to 3000 mg are possible, for example, from about 325 mg to about 475 mg, from about 350 mg to about 500 mg, from about 375 to about 525 mg, from about 400 mg to about 550 mg, from about 425 mg to about 575 mg, from about 450 mg to about 600 mg, from about 475 mg to about 625 mg, from about 500 mg to about 650 mg, from about 525 mg to about 675 mg, from about 550 mg to about 700 mg, from about 575 mg to about 725 mg, from about 600 mg to about 750 mg, from about 625 mg to about 775 mg, from about 650 mg to about 800 mg, from about 675 mg to about 825 mg, from about 700 mg to about 850 mg, from about 725 mg to about 875 mg, from about 750 mg to about 900 mg, from about 775 mg to about 925 mg, from about 800 mg to about 950 mg, from about 825 to about 975, from about 850 mg to about 1000 mg, from about 900 mg to about 1150 mg, from about 1000 mg to about 1150 mg, from about 1100 mg to about 1250 mg, and from about 1200 mg to about 1350 mg.

“wt %” means weight percent based on the total weight of the composition or formulation.

The compositions of the present disclosure can comprise from about 10 mg to about 3000 mg of the one or more spirocyclic compounds disclosed herein, and one or more pharmaceutically acceptable excipients. The compositions of the present disclosure can comprise 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg, 1050 mg, 1100 mg, 1150 mg, 1200 mg, 1250 mg, 1300 mg, 1350 mg, and 1400 mg of the one or more spirocyclic compounds disclosed herein, and, additionally, one or more pharmaceutically acceptable excipients.

Compositions for inhalation or insufflation include solutions and suspensions in pharmaceutically acceptable aqueous or organic solvents, mixtures thereof, and powders. Dosage ranges and composition weights for inhalation or insufflation are as disclosed above.

Dosage forms for topical or transdermal administration of a subject composition include powders, sprays, ointments, pastes, creams, lotions, gels, solutions, and patches.

It should be understood that the foregoing description is only illustrative of the present disclosure. Various alternatives and modifications can be devised by those skilled in the art without departing from the present disclosure. Accordingly, the present disclosure is intended to embrace all such alternatives, modifications and variances which fall within the scope of the appended claims.

1. A method of treating or preventing sarcoidosis-associated pulmonary hypertension in a human patient comprising administering to the patient a therapeutically effective amount of about 1 mg/kg/day to about 50 mg/kg/day of a compound selected from the group consisting of (i) (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate or a pharmaceutically acceptable salt thereof, (ii) (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid or a pharmaceutically acceptable salt thereof, and (iii) a combination of the foregoing.

2. The method of claim 1, wherein the compound or pharmaceutically acceptable salt thereof is administered orally.

3. The method of claim 1, wherein the compound or pharmaceutically acceptable salt thereof is administered orally by a dosage form selected from the group consisting of capsules, tablets, powders, and granules.

4. The method of claim 1, wherein the compound or pharmaceutically acceptable salt thereof is administered orally in the form of a liquid.

5. The method of claim 1, wherein the compound or pharmaceutically acceptable salt thereof is administered one to four times per day.

6. The method of claim 1, wherein the compound is (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate or a pharmaceutically acceptable salt thereof.

7. The method of claim 1, wherein the compound is (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate.

8. The method of claim 2, wherein the compound is (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate or a pharmaceutically acceptable salt thereof.

9. The method of claim 2, wherein the compound is (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate.

10. The method of claim 3, wherein the compound is (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate.

11. The method of claim 1, wherein the compound is (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid or a pharmaceutically acceptable salt thereof.

12. The method of claim 1, wherein the compound is (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid.

13. The method of claim 1, wherein the compound is in a substantially amorphous form.

14. The method of claim 1, wherein the compound is in a substantially crystalline form.

15. The method of claim 11, wherein the compound is a crystalline polymorph having a XRPD plot corresponding to FIG. 1.

16. The method of claim 11, wherein the compound is a crystalline polymorph having a XRPD plot corresponding to Table 1 or Table 2.

17. The method of claim 11, wherein the compound is a crystalline polymorph exhibiting a characteristic XRPD peak at 19.05±0.20 (°2θ).

18. The method of claim 1, wherein the compound or pharmaceutically acceptable salt thereof is administered systemically.

19. The method of claim 1, wherein the compound or pharmaceutically acceptable salt thereof is administered via inhalation or insufflation.

20. A method of treating sarcoidosis-associated pulmonary hypertension in a human patient, comprising administering to the human patient a composition including
a therapeutically effective amount of about 1 mg/kg/day to about 50 mg/kg/day of a compound selected from the group consisting of (i) (S)-ethyl 8-(2-amino-6-((R)-1-(5-chloro-[1,1′-biphenyl]-2-yl)-2,2,2-trifluoroethoxy)pyrimidin-4-yl)-2,8-diazaspiro[4.5]decane-3-carboxylate or a pharmaceutically acceptable salt thereof, (ii) (S)-8-(2-amino-6-((R)-1-(5-chloro-[1,1′-biphenyl]-2-yl)-2,2,2-trifluoroeth-oxy)pyrimidin-4-yl)-2,8-diazaspiro[4.5]decane-3-carboxylic acid or a pharmaceutically acceptable salt thereof, and (iii) a combination of the foregoing; and
a pharmaceutically acceptable excipient.
